(19) United States
(12) Patent Application Publication
(10) Pub. No.: US 2023/0200723 A1
(43) Pub. Date: Jun. 29, 2023

(54) WOUND MANAGEMENT SYSTEM FOR PREDICTING AND TREATING WOUNDS

(71) Applicant: MatrixCare, Inc., Bloomington, MN (US)

(72) Inventors: Jessica ROCKNE, Bloomington, MN (US); Vivek Kumar, Bloomington, MN (US); Adhiraj Ganpat Prajapati, Bloomington, MN (US); Robert Price, Bloomington, MN (US); Kedar Mangesh Kadam, Bloomington, MN (US); Timothy James Heeren, Bloomington, MN (US); Nitin Gandhi, Bloomington, MN (US); Coleen Patrice Danielson, Bloomington, MN (US)

(21) Appl. No.: 17/562,771

(22) Filed: Dec. 27, 2021

(51) Int. Cl. A61B 5/00

(52) U.S. Cl. CPC A61B 5/445; A61B 5/7275; A61B 5/7267

(57) ABSTRACT

Certain aspects of the present disclosure provide a wound management system and method for predicting and treating wounds. The method includes collecting data relating to a patient's health and applying a machine learning model to the data relating to the patient's health to predict a first probability that the patient will sustain a first wound type outside of a care setting. The method also includes, in response to determining that the first probability exceeds a threshold, determining an action that reduces the first probability and communicating, to the patient, a message indicating the action should be taken to reduce the first probability that the patient will sustain the first wound type.

### INTRODUCTION

Aspects of the present disclosure relate to a wound management system for predicting and treating wounds. A focus of the healthcare industry is the treatment of wounds. The conventional approach towards wound treatment, however, is reactive: the patient does not receive care until the patient actually sustains the wound. As a result, there are techniques for treating and healing many different types of wounds, but the incidences or occurrences of wounds is not necessarily decreasing.

Additionally, conventional processes for determining whether a patient is likely to sustain a wound outside a care setting relied on subjective human judgment and analysis of the patient's information, which resulted in an incomplete analysis of the patient's information and inaccurate predictions. Recommendations and actions taken based on these inaccurate predictions may not actually improve the health and well-being of the patient or reduce the incidences or occurrences of wounds.

### SUMMARY

A wound management system and method for predicting and treating wounds are described herein. According to an embodiment, a method includes collecting data relating to a patient's health and applying a machine learning model to the data relating to the patient's health to predict a first probability that the patient will sustain a first wound type outside of a care setting. The method also includes, in response to determining that the first probability exceeds a threshold, determining an action that reduces the first probability and communicating, to the patient, a message indicating the action should be taken to reduce the first probability that the patient will sustain the first wound type. Other embodiments include an apparatus and a processing system that perform this method. Additional embodiments include a non-transitory computer-readable medium and a computer program product that include instructions that, when executed by a processor, cause the processor to perform this method.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 illustrates an example system.

FIG. 2 illustrates an example wound management system in the system of FIG. 1.

FIG. 3 illustrates example health data in the system of FIG. 1.

FIG. 4 illustrates example health screening data in the system of FIG. 1.

FIG. 5 illustrates an example wound management system in the system of FIG. 1.

FIG. 6 illustrates an example wound management system in the system of FIG. 1.

FIG. 7 illustrates an example operation in the system of FIG. 1.

FIG. 8 is a flowchart of an example method performed in the system of FIG. 1.

FIG. 9 is a flowchart of an example method performed in the system of FIG. 1.

FIG. 10 is a flowchart of an example method performed in the system of FIG. 1.

FIG. 11 illustrates an example device in the system of FIG. 1.

FIG. 12 illustrates an example device in the system of FIG. 1.

FIG. 13 illustrates an example device in the system of FIG. 1.

FIG. 14 is a flowchart of an example method performed in the system of FIG. 1.

FIG. 15 illustrates an example device in the system of FIG. 1.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer readable mediums for predicting and treating wounds sustained outside a care setting. Specifically, this disclosure describes a wound management system that uses machine learning to predict whether a patient is likely to sustain different wound types outside of a care setting based on information about the patient's life. For example, the wound management system may predict that a patient is more likely to sustain cuts or burns at work if the patient is a chef. As another example, the wound management system may predict that the patient is more likely to sustain wounds from falling if the patient enjoys rock climbing. The wound management system may also prevent the predicted wound types from occurring by recommending actions that the patient can take to reduce the likelihood of sustaining the wound types. In this manner, the wound management system provides a proactive approach towards wound treatment, which improves the health and well-being of the patient, in certain embodiments.

### Example Systems and Methods

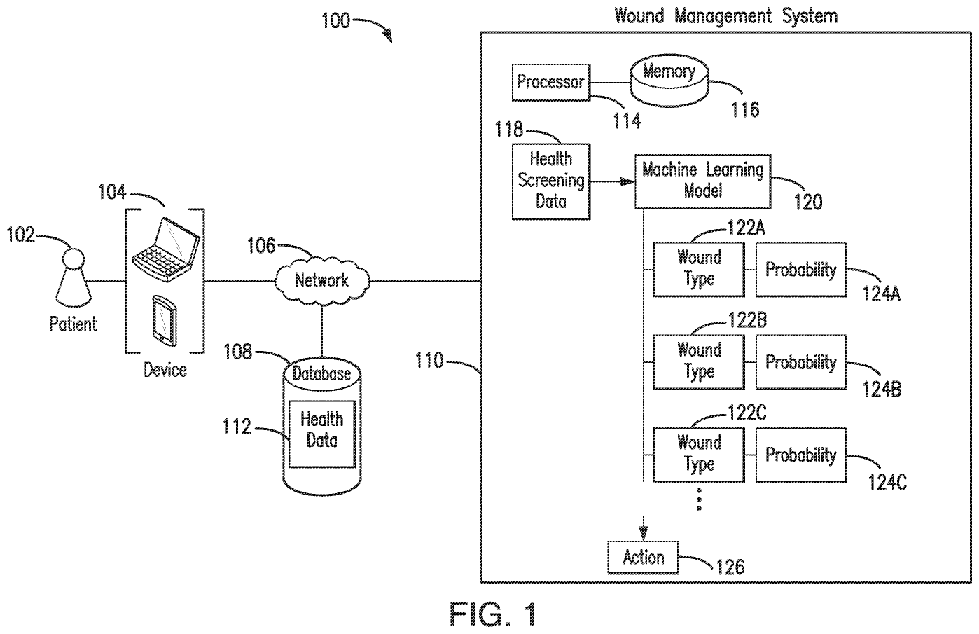

FIG. 1 illustrates an example system 100. As seen in FIG. 1, the system 100 includes one or more devices 104, a network 106, a database 108, and a wound management system 110. Generally the system 100 applies one or more machine learning models to information about a patient's 102 life (e.g., the patient's 102 demographics, career, and habits) to predict how likely the patient 102 is to sustain different types of wounds. The system 100 proactively addresses these likelihoods by providing warnings to the patient 102 or by recommending remedial actions to be taken by the patient 102. As a result, the system 100 reduces the likelihood that the patient 102 will sustain different wound types, which improves the health and wellbeing of the patient 102 and reduces the incidences or occurrences of wounds, in particular embodiments.

Specifically, conventional processes for determining whether a patient 102 is likely to sustain a wound outside a care setting relied on subjective human judgment and analysis of the patient's 102 information, which resulted in an incomplete analysis of the patient's 102 information and inaccurate predictions. Recommendations and actions taken based on these inaccurate predictions may not actually improve the health and well-being of the patient 102 or reduce the incidences or occurrences of wounds. Additionally, the subjective human assessments often involved bias, which resulted in inconsistent predictions and recommendations. The wound management system 110, on the other hand, applies a machine learning model to the patient's 102 information to perform a complete analysis of the patient's 102 information, which provides the technical advantage of a more accurate prediction of the likelihood that the patient 102 will sustain a wound outside a care setting. The wound management system 110 also provides recommendations based on these more accurate predictions, which effects a particular treatment or prophylaxis for preventing or reducing the likelihood of sustaining wounds outside a care setting. By using machine learning to predict and treat wounds, the wound management system 110 significantly reduces human subjectivity, which overcomes bias and increases consistency.

The patient 102 uses the device 104 to provide information about the patient 102. For example, the patient 102 may be at a healthcare facility. During the check-in process, the patient 102 responds to a questionnaire or survey that asks for information about the patient 102. After this information is collected, the system 100 analyzes this information to determine how likely it is for the patient 102 to sustain different wound types. As another example, the patient 102 may be using a personal device 104 at home or at work to execute an application. The patient 102 responds to a survey or questionnaire presented by the application to provide information about the patient 102. After the information is collected, the system 100 analyzes that information to predict how likely it is for the patient 102 to sustain different types of wounds.

The device 104 is any suitable device for communicating with components of the system 100 over the network 106. As an example and not by way of limitation, the device 104 may be a computer, a laptop, a wireless or cellular telephone, an electronic notebook, a personal digital assistant, a tablet, or any other device capable of receiving, processing, storing, or communicating information with other components of the system 100. The device 104 may be a wearable device such as a virtual reality or augmented reality headset, a smart watch, or smart glasses. The device 104 may also include a user interface, such as a display, a microphone, keypad, or other appropriate terminal equipment usable by the patient 102. The device 104 may include a hardware processor, memory, or circuitry that perform any of the functions or actions of the device 104 described herein. For example, a software application designed using software code may be stored in the memory and executed by the processor to perform the functions of the device 104.

The network 106 is any suitable network operable to facilitate communication between the components of the system 100. The network 106 may include any interconnecting system capable of transmitting audio, video, signals, data, messages, or any combination of the preceding. The network 106 may include all or a portion of a public switched telephone network (PSTN), a public or private data network, a local area network (LAN), a metropolitan area network (MAN), a wide area network (WAN), a local, regional, or global communication or computer network, such as the Internet, a wireline or wireless network, an enterprise intranet, or any other suitable communication link, including combinations thereof, operable to facilitate communication between the components.

The database 108 stores information about previously sustained wounds. As seen in FIG. 1, the database 108 stores health data 112. The health data 112 may include information about other individuals and the wounds they have previously sustained. For example, the health data 112 may include information such as the demographics, home conditions, work conditions, symptoms, and habits of the other individuals. Additionally, the health data 112 may include the wound types sustained by these other individuals and the times at which the wound types were sustained. The system 100 uses the health data 112 to train a machine learning model to predict how likely it is that the patient 102 will sustain certain wound types outside of a care setting. For example, the machine learning model may analyze the health data 112 to detect patterns or trends in the demographics and lifestyles of the other individuals that may result in particular wound types being sustained. Once trained, the machine learning model may then analyze information about the patient 102 to determine whether the patterns or trends also exist in the lifestyle of the patient 102. The machine learning model then predicts the likelihood that the patient 102 will sustain different wound types based on these detected patterns or trends.

The wound management system 110 collects information about the patient 102 and applies a machine learning model to that information to predict how likely it is that the patient 102 will sustain different wound types. Additionally, the wound management system 110 provides warnings or remedial actions that the patient 102 can take to reduce the likelihood that the patient 102 will sustain the wound types. In some embodiments, the wound management system 110 is a computer system (e.g., a server) separate from the device 104. In some embodiments the wound management system 110 is embodied within the device 104. For example, the device 104 may implement the wound management system 110 by executing an application on the device 104. As seen in FIG. 1, the wound management system 110 includes a processor 114 and a memory 116, which may perform the actions or functions of the wound management system 110 described herein. In embodiments where the wound management system 110 is embodied within the device 104, the processor 114 and the memory 116 may be the processor and memory of the device 104.

The processor 114 is any electronic circuitry, including, but not limited to one or a combination of microprocessors, microcontrollers, application specific integrated circuits (ASIC), application specific instruction set processor (ASIP), and/or state machines, that communicatively couples to memory 116 and controls the operation of the wound management system 110. The processor 114 may be 8-bit, 16-bit, 32-bit, 64-bit or of any other suitable architecture. The processor 114 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The processor 114 may include other hardware that operates software to control and process information. The processor 114 executes software stored on the memory 116 to perform any of the functions described herein. The processor 114 controls the operation and administration of the wound management system 110 by processing information (e.g., information received from the devices 104, network 106, and memory 116). The processor 114 is not limited to a single processing device and may encompass multiple processing devices.

The memory 116 may store, either permanently or temporarily, data, operational software, or other information for the processor 114. The memory 116 may include any one or a combination of volatile or non-volatile local or remote devices suitable for storing information. For example, the memory 116 may include random access memory (RAM), read only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 116, a disk, a CD, or a flash drive. In particular embodiments, the software may include an application executable by the processor 114 to perform one or more of the functions described herein.

The wound management system 110 collects health screening data 118 from the patient 102 or the device 104. The health screening data 118 may be provided by the patient 102 in response to, for example, questionnaires or surveys. In some embodiments, the wound management system 110 communicates these questionnaires or surveys to the device 104. The patient 102 responds to the surveys or questionnaires using the device 104. The device 104 then communicates these responses back to the wound management system 110 as the health screening data 118. The health screening data 118 may include any suitable information about the patient 102. For example, the health screening data 118 may include demographics information about the patient 102 (e.g., age, gender, and location). As another example, the health screening data 118 may include information about the lifestyle of the patient 102 (e.g., home conditions, work conditions, habits, or hobbies). The health screening data 118 may also include medical information of the patient 102 (e.g., allergies, vaccinations, hospitalizations, operations, medications, and a family medical history). The wound management system 110 collects and analyzes the health screening data 118 to predict the likelihood that the patient 102 will sustain various wound types.

The wound management system 110 applies a machine learning model 120 to the health screening data 118 to detect patterns or trends in the health screening data 118. Detected patterns or trends are then used to determine how likely it is that the patient 102 will sustain different wound types. As seen in FIG. 1, the machine learning model 120 analyzes the health screening data 118 to determine probabilities 124 that the patient 102 will develop different wound types 122. In the example of FIG. 1, the machine learning model 120 analyzes the health screening data 118 and determines that the patient 102 has a probability 124A of sustaining a wound type 122A, a probability 124B of sustaining the wound type 122B, and the probability 124C of sustaining the wound type 122C.

The wound management system 110 may implement supervised machine learning techniques, unsupervised machine learning techniques, or a combination of supervised and unsupervised learning techniques. In an embodiment, a user or an administrator selects the machine learning model to apply based on knowledge or analysis of the health screening data 118 or the health data 112. For example, the wound management system 110 may begin by applying logistic regression to the health screening data 118 or the health data 112. After that analysis is complete, the user or administrator may select another machine learning model to apply that the user or administrator is more suitable for the data. In some embodiments, the wound management system 110 analyzes the health screening data 118 or the health data 112 and determines a machine learning model to apply to the data based on that analysis. Whether supervised or unsupervised techniques are used, the wound management system 110 may convert the health screening data 118 or the health data 112 to a numerical format, and based on an initial data analysis, data transformation techniques may be chosen (e.g., by the wound management system 110 or by a user or administrator).

Each wound type 122 may be a category that encompasses many different wounds. For example, a wound type 122 of “wounds sustained from falling” may encompass wounds such as lacerations, bruises, breaks, scrapes, and contusions. As another example, a wound type 122 of “self-inflicted wounds” may encompass wounds such as cuts, burns, and scratches. As yet another example, a wound type 122 of “wounds sustained from ambulatory conditions” may encompass wounds such as contusions, breaks, and scrapes. The wound management system 110 may determine the likelihood that a patient 102 will develop certain wound types 122 and provide warnings or recommendations to the patient to reduce that likelihood.

The wound management system 110 determines one or more actions 126 based on the probabilities 124. For example, if a probability 124 that the patient 102 will sustain a particular wound type 122 is high, the wound management system 110 may determine a remedial action that the patient 102 may take to reduce that probability 124. As another example, if a probability 124 that the patient 102 will sustain a particular wound type 122 is low, the wound management system 110 may provide a general warning to the patient 102 of the risks that the patient 102 will sustain that wound type 122. In certain embodiments, a database or repository may store actions 126 that should be recommended to remedy or avoid certain wound types 122. The wound management system 110 determines the one or more actions 126 by querying the database or repository using the wound type 122. The database or repository then returns the one or more actions 126. For example, as healthcare facilities treat patients that sustained wounds, the healthcare facilities may log, in the database 108 or as part of the health data 112, the wound types sustained by the patients and the treatments or remedies that were recommended to the patients 102 for avoiding those wound types in the future. When the wound management system 110 queries the database 108 using the determined wound type 122, the database 108 may return the one or more actions 126 based on the treatments and remedies previously recommended for that wound type 122.

The wound management system 110 may recommend any suitable actions 126. For example, the wound management system may recommend that a patient 102 wear different types of protective clothing (e.g., gloves and boots) while working to protect against certain wound types 122. As another example, the wound management system may recommend that a patient 102 change what and when the patient 102 eats or that a patient 102 change careers or hobbies to reduce the likelihood of sustaining certain wound types 122. As yet another example, the wound management system may recommend that the patient 102 visit a particular healthcare facility if the patient 102 sustains a certain wound type. The wound management system 110 may use an address in the health screening data 118 to identify healthcare facilities near the patient 102. The wound management system 110 may then examine treatment statistics for these healthcare facilities (e.g., statistics in the health data 112) to identify and recommend the healthcare facility that is most successful or best suited for treating the particular wound type.

The wound management system 110 communicates a message to the patient 102 or the device 104 that indicates the one or more actions 126 determined by the wound management system 110. When the patient 102 sees the message, the patient 102 may implement the remedial actions or heed the warnings provided by the wound management system 110. As a result, the wound management system 110 effects a particular treatment or prophylaxis that prevents or reduces the likelihood that the patient 102 will sustain the wound types 122, which improves the health and wellbeing of the patient 102 and reduces the incidences and occurrences of the wound types 122, in certain embodiments.

In some embodiments, the wound management system 110 may detect changes in the patient's 102 life or in the health screening data 118. For example, the wound management system 110 may detect, based on the location of the device 104, that the patient 102 is traveling to different places associated with different careers or hobbies. As another example, the wound management system 110 may detect, based on a social media feed of the patient 102 that the patient 102 changed career or hobbies. As yet another example, the patient 102 may use the device 104 to let the wound management system 110 know that the patient 102 has changed careers or hobbies. In response to detecting these changes, the wound management system 110 re-applies the machine learning model 120 to the updated data to reassess the likelihood that the patient 102 will sustain certain wound types 122. The wound management system 110 may then provide updated actions 126 that the patient 102 may take to reduce these updated likelihoods.

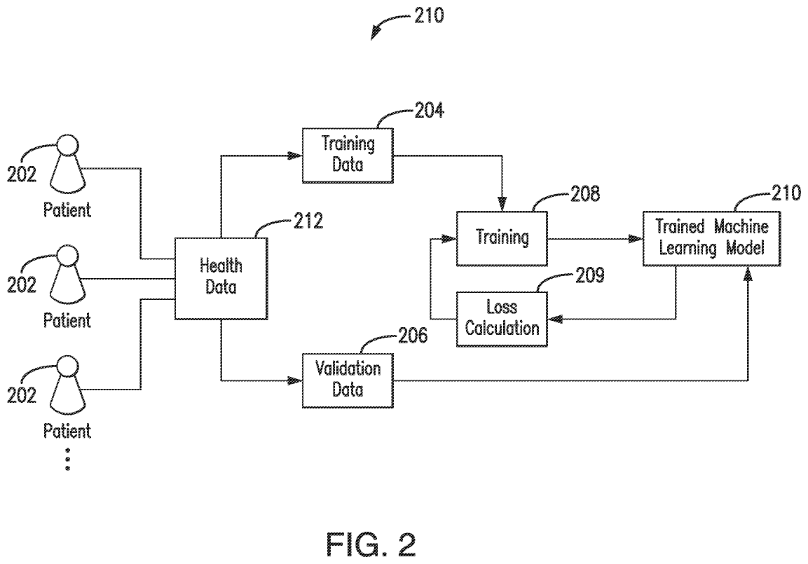

FIG. 2 illustrates an example wound management system 110 in the system 100 of FIG. 1. Generally, FIG. 2 shows the wound management system 110 training a machine learning model. Although the wound management system 110 is shown training the machine learning model, it is contemplated that a computer system separate from the wound management system 110 may train the machine learning model and then deploy the machine learning model to the wound management system 110.

The wound management system 110 begins by collecting the health data 112 from one or more patients 202. For example, as the patients 202 sustained wounds and are treated at healthcare facilities, the healthcare facilities may collect information about the patients 202. This information may include the demographics of the patients 202, the careers and habits of the patients 202, and the medical history of the patients 202. Additionally, the healthcare facilities may log information about the wounds sustained by the patients 202 and the times that those wounds were sustained. Moreover, the healthcare facilities may log the treatments and remedies that were prescribed or recommended to the patients 202. All of this information may be encapsulated within the health data 112. The health data 112 may then be stored in the database 108 shown in FIG. 1. When the wound management system 110 (or another computer system) is ready to train the machine learning model, the wound management system 110 may retrieve the health data 112 from the database 108. The wound management system 110 then uses the health data 112 to train the machine learning model.

The wound management system 110 splits or divides the health data 112 into two datasets. In the example of FIG. 2, the wound management system 110 splits the health data 112 into training data 204 and validation data 206. The wound management system 110 may use any suitable process for splitting the health data 112 into the training data 204 and the validation data 206. For example, the wound management system 110 may analyze the health data 112 and select the datapoints that are most different from each other to form the training data 204. The remaining datapoints then form the validation data 206. As another example, the wound management system 110 may cluster the health data 112 such that the datapoints in the health data 112 that are most similar to each other are assigned to the same cluster. The wound management system 110 then selects datapoints from different clusters to form the training data 204, which may ensure that the training data 204 includes a diverse set of datapoints. The remaining datapoints then form the validation data 206. In this manner, the wound management system 110 may use a diverse set of datapoints to train the machine learning model, which increases the robustness, generalizability, and accuracy of the machine learning model, in particular embodiments.

The wound management system 110 trains the machine learning model (in a block 208) by having the machine learning model analyze the training data 204 to detect patterns or trends in the training data 204. Through this training, the machine learning model learns to predict the likelihood that a particular wound type will be sustained based on detected patterns or trends. The wound management system 110 then uses the validation data 206 to validate the trained machine learning model 210. For example, the wound management system 110 may apply the trained machine learning model 210 to the validation data 206 to see if the trained machine learning model 210 makes accurate predictions. During this validation process, the wound management system 110 performs a loss calculation 209, which indicates an accuracy or loss of the machine learning model. If the loss or accuracy of the trained machine learning model 210 is not acceptable, the wound management system 110 may perform another round of training 208. This cycle of training and validation may continue until the loss or accuracy of the machine learning model improves to an acceptable level.

In certain embodiments, the loss may be calculated by applying the trained machine learning model 210 to datapoints within the validation data 206. The trained machine learning model 210 may detect a pattern or trend in the validation data 206 and predict a likelihood that particular wound types will be sustained based on the detected patterns or trends. The wound management system 110 then compares the predicted likelihood against the actual wounds sustained indicated by the validation data 206. The difference in the predicted likelihood and the actual sustained wounds represents the loss or accuracy of the trained machine learning model 210. The wound management system 110 may perform additional iterations of training until this loss or accuracy is at an acceptable level.

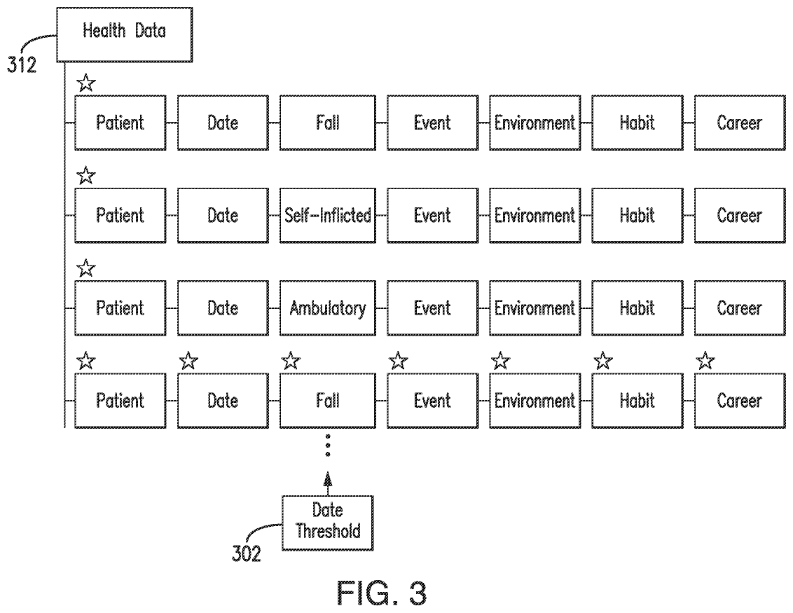

FIG. 3 illustrates example health data 112 in the system 100 of FIG. 1. As discussed previously, the health data 112 may include information about previous patients 202 and the wounds sustained by these previous patients 202.

As seen in FIG. 3, the health data 112 includes information that identifies the patients 202 (e.g., names and addresses). In certain embodiments, the wound management system 110 removes or excludes this patient information (indicated by an ‘*’) from the health data 112 before using the health data 112 to train a machine learning model. In this manner, the wound management system protects the privacy and security of the previous patients 202.

The health data 112 also includes information about the wounds sustained by the previous patients 202. The health data 112 may include the dates on which the wounds were sustained and the wound types of the particular wounds. As seen in FIG. 3, each of the datapoints includes a date field that indicates the date on which a wound was sustained. Additionally, each datapoint includes a wound type such as a wound sustained from a fall, a self-inflicted wound, or a wound sustained through ambulatory conditions.

The health data 112 may also include information about the context in which the wound was sustained. As seen in FIG. 3, the health data 112 may include an event, which may indicate an action or activity in which the patient 202 was engaging when the wound was sustained. For example, the event may include cooking, climbing, running, driving, playing sports, etc. Additionally, the health data 112 may include an environment, which indicates the setting in which the wound was sustained. For example, the environment may include home, work, gym, kitchen, park, lake, mountains, etc. The machine learning model may use this information to determine what kinds of events and environments cause certain wounds to be sustained.

The health data 112 may also include information about the lifestyle of the patients 202. As seen in FIG. 3, the health data 112 includes information about the habits and careers of the patients 202. The habits may indicate the activities and practices in which the patients 202 regularly engage. The careers may indicate the jobs that the patients 202 hold. The machine learning model may use this information to determine the habits and careers that are likely to result in certain wound types being sustained.

In some embodiments, the wound management system 110 may exclude or remove certain information from the health data 112 based on the date that a wound was sustained (indicated by an ‘*’). For example, the wound management system 110 may compare the date from each datapoint in the health data 112 with a date threshold 302. If the date is earlier than the date threshold 302, then the wound management system 110 may remove the datapoint with that date from the health data 112 so that the machine learning model is not trained using that datapoint. As a result, the wound management system 110 removes datapoints that are old from being used to train the machine learning model, which may improve the accuracy of the machine learning model, in particular embodiments.

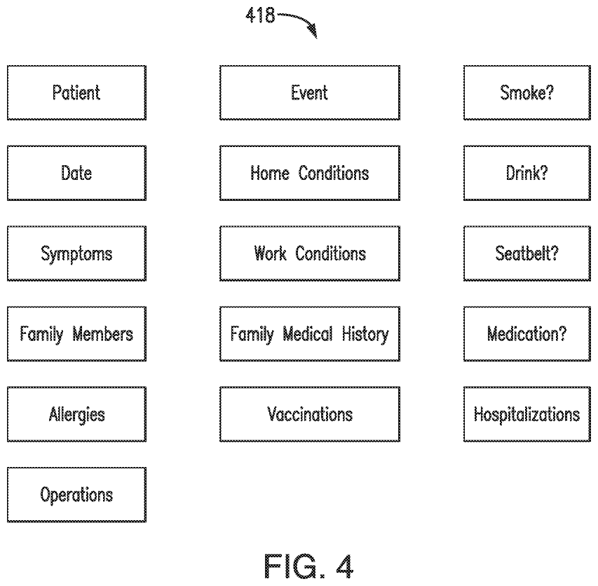

FIG. 4 illustrates example health screening data 118 in the system 100 of FIG. 1. A patient 102 may provide the health screening data 118 using a device 104 when the patient 102 is checking in to a healthcare facility. Additionally or alternatively, the patient 102 may provide the health screening data 118 by executing an application on a personal device 104 of the patient 102 (e.g., at work or at home). The patient 102 may provide the information by responding to a questionnaire or survey. The responses to the questionnaire or survey provide the information in the health screening data 118. After the device 104 collects the responses, the device 104 communicates the responses to the wound management system 110 as the health screening data 118.

As seen in FIG. 4, the health screening data 118 includes information that identifies the patient 102. For example, the health screening data 118 may include the name, address, and birthdate of the patient 102 as well as information that identifies the family members of the patient 102. This information may be needed to properly store the health screening data 118 and to properly notify the patient 102 of any health or medical developments.

The health screening data 118 also includes medical information about the patient 102. For example, the health screening data 118 may indicate the allergies that the patient 102 has and the vaccinations that the patient 102 has received. Additionally, the health screening data 118 may indicate the previous hospitalizations of the patient 102 as well as previous operations that the patient 102 has received. The health screening data 118 may also indicate the medications that are taken or have been taken by the patient 102. The health screening data 118 may also include information about symptoms experienced by the patient 102. In some instances, the health screening data 118 also includes a family medical history for the patient 102, which indicates medical conditions that family members of the patient 102 have had (e.g., heart attacks or strokes).

The health screening data 118 also includes information about the lifestyle of the patient 102. For example, the health screening data 118 may include information about the home conditions or work conditions of the patient 102. The home conditions may indicate whether the home of the patient 102 is cluttered, includes stairs, or has carbon monoxide detectors. The work conditions may indicate the career of the patient 102 as well as descriptions of the duties and responsibilities of the patient 102 at work. For example, the work conditions may indicate that the patient 102 is a chef who prepares and cooks food regularly in a large kitchen. As another example, the work conditions may indicate that the patient 102 is a utility maintenance provider who regularly climbs utility poles. The health screening data 118 may also include event information that indicates various activities or practices in which the patient 102 engages. For example, the health screening data 118 may indicate that the patient 102 enjoys rock climbing or racecar driving.

The health screening data 118 also includes information about the habits of the patient 102. For example, the health screening data 118 may include information that indicates whether the patient 102 smokes or drinks. Additionally, the health screening data 118 may include information that indicates whether the patient 102 wears a seatbelt or uses recreational drugs.

The wound management system 110 may apply the machine learning model 120 to the information within the health screening data 118 to determine likelihoods that the patient 102 will sustain particular wound types. For example, the machine learning model 120 may predict that the patient 102 is more likely to sustain particular types of wounds as a result of the patient's career. As another example, the machine learning model 120 may predict that the patient 102 is more likely to sustain particular wound types due to the patient's 102 habits. Based on these predictions, the wound management system 110 may proactively warn the patient 102 or recommend remedial actions to the patient 102 that may reduce the likelihood that the patient 102 will sustain these wound types, in particular embodiments.

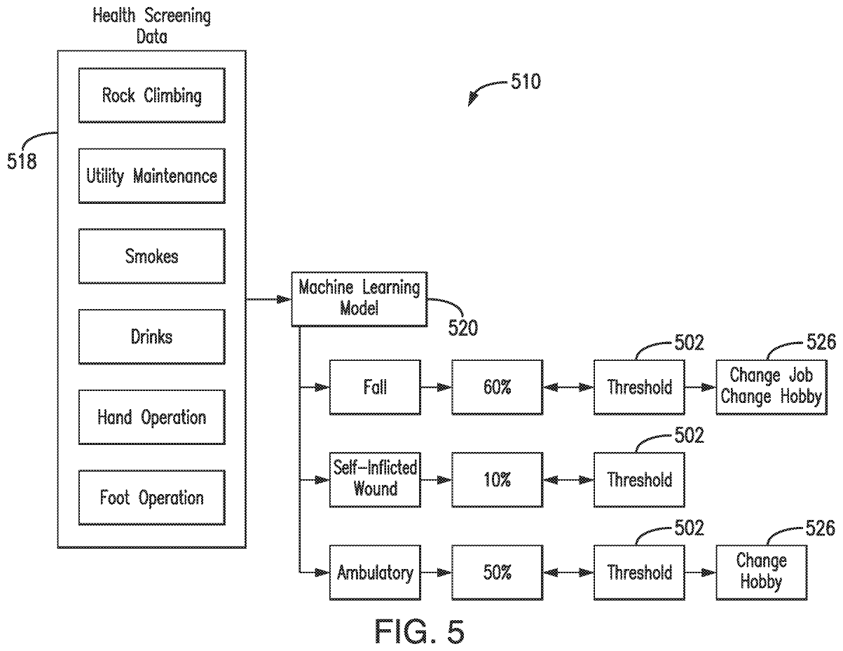

FIG. 5 illustrates an example wound management system 110 in the system 100 of FIG. 1. Generally, FIG. 5 shows the wound management system 110 applying the machine learning model 120 to the health screening data 118 of a utility maintenance provider. The machine learning model 120 predicts that the utility maintenance provider is likely to sustain particular wound types, and the wound management system 110 provides warnings and recommendations to the utility maintenance provider based on these predictions.

The health screening data 118 indicates that the utility maintenance provider has previously had hand and foot operations. Additionally, the health screening data 118 indicates that the utility maintenance provider enjoys rock climbing but also smokes and drinks. The utility maintenance provider may have provided this health screening data 118 while checking into a healthcare facility. Alternatively, the utility maintenance provider may have provided the health screening data 118 while at home using a personal device 104 of the utility maintenance provider.

The wound management system 110 applies the machine learning model 120 to the information within the health screening data 118 to detect patterns and trends within the health screening data 118. For example, the machine learning model 120 may identify previous patients 202 that are similar to the utility maintenance provider by comparing the health screening data 118 to the health data 112 of the previous patients 202. In the example of FIG. 5, the machine learning model 120 determines, based on the health screening data 118, that the utility maintenance provider has a 60% chance of sustaining a wound from a fall, a 10% chance of sustaining a self-inflicted wound, and a 50% chance of sustaining a wound from ambulatory conditions.

After the machine learning model 120 determines the likelihood that the utility maintenance provider will sustain particular wound types, the wound management system 110 analyzes the predicted likelihoods to determine if any remedial action is appropriate. The wound management system 110 compares each of the determined likelihoods to a threshold 502. If the determine likelihood exceeds the threshold 502, then the wound management system 110 may determine a remedial action 126 that the utility maintenance provider may take to reduce the likelihood of sustaining a particular wound type corresponding to that likelihood. In the example of FIG. 5, the wound management system compares the likelihood that the utility maintenance provider will sustain a wound from a fall to the threshold 502. The wound management system 110 may determine that the 60% likelihood exceeds the threshold 502. In response, the wound management system 110 determines actions 126 that include changing jobs or changing hobbies. Stated differently, the wound management system 110 may determine that the utility maintenance provider has a high likelihood of sustaining wounds from a fall due to the fact that the utility maintenance provider enjoys rock climbing or climbs utility poles for a living. The wound management system 110 may determine that this likelihood may be reduced if the utility maintenance provider changes jobs or finds a new hobby.

The wound management system 110 compares the likelihood that the utility maintenance provider will sustain a self-inflicted wound with the threshold 502. The wound management system 110 may determine that the 10% likelihood is below the threshold 502. In response, the wound management system 110 may not determine a remedial action to recommend to the utility maintenance provider. In some embodiments the wound management system 110 may still provide a warning to the utility maintenance provider that the utility maintenance provider has a risk of sustaining a self-inflicted wound (e.g., while setting up climbing gear for work or for fun).

The wound management system 110 compares the likelihood that the utility maintenance provider will sustain a wound from ambulatory conditions to the threshold 502. The wound management system 110 may determine that the 50% likelihood exceeds the threshold 502. In response, the wound management system 110 determines an action 126 that the utility maintenance provider can take to reduce the likelihood. In the example of FIG. 5, the wound management system 110 determines that the utility maintenance provider may reduce the risk of sustaining a wound due to ambulatory conditions by finding a new hobby. After assessing each of the predicted likelihoods, the wound management system 110 may communicate the determined actions 126 to the utility maintenance provider. The utility maintenance provider may take the recommended actions 126 to reduce the predicted likelihoods of sustaining the various wound types.

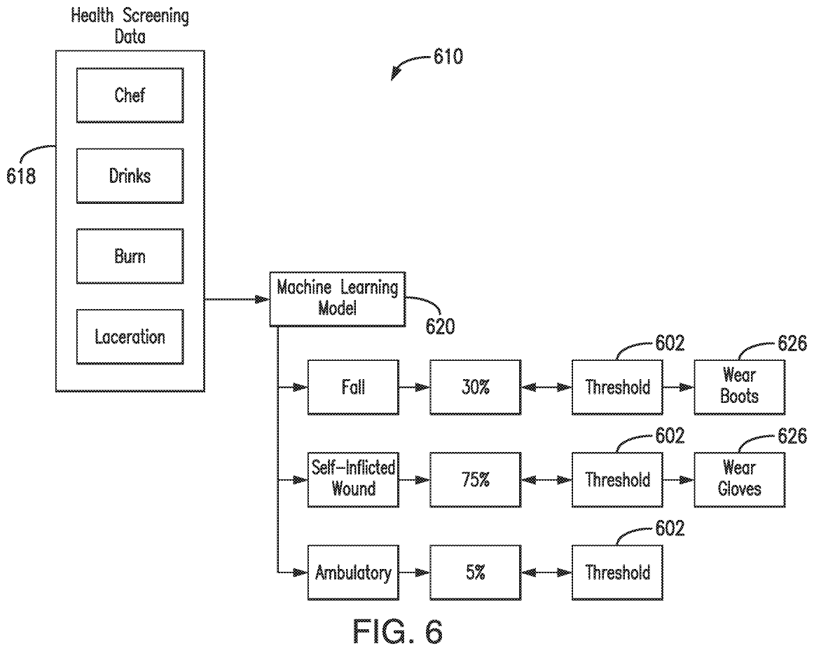

FIG. 6 illustrates an example wound management system 110 in the system 100 of FIG. 1. Generally, FIG. 6 shows the wound management system 110 applying the machine learning model 120 to health screening data 118 of a chef. The machine learning model 120 predicts likelihoods that the chef will sustain particular wound types. The wound management system 110 then analyzes these predicted likelihoods to determine remedial actions 126 that the chef can take to reduce these likelihoods, which improves the health and wellbeing of the chef in certain embodiments.

The wound management system 110 collects the health screening data 118 from the chef. The chef may have provided the health screening data 118 when checking into a healthcare facility. Alternatively, the chef may have provided the health screening data 118 using the chef s personal device 104 while at work or at home. In the example of FIG. 6, the health screening data 118 indicates a career of chef. Additionally, the health screening data 118 indicates that the chef has a history of suffering burns and lacerations. Additionally, the health screening data 118 indicates that the chef drinks. The wound management system 110 may collect and analyze the health screening data 118 to determine wound types that the chef is likely to sustain.

The wound management system 110 applies the machine learning model 120 to the health screening data 118 to determine the likelihoods. The machine learning model 120 may then analyze the health data 112 of these other patients 202 to determine the likelihood that the chef will sustain certain wound types. In the example of FIG. 6, the machine learning model 120 determines that the chef has a 30% chance of sustaining a wound from a fall, a 75% chance of sustaining a self-inflicted wound and a 5% chance of sustaining a wound due to ambulatory conditions. For example, the machine learning model 120 may determine that the chef has a 30% chance of slipping or falling while working on a wet floor in a kitchen. As another example, the machine learning model 120 may determine that the chef has a 75% chance of sustaining a burn or cut while working in the kitchen.

The wound management system 110 compares the predicted likelihoods with the threshold 502 to determine whether a remedial action 126 should be recommended. In the example of FIG. 6, the wound management system 110 compares the 30% chance of sustaining a wound due to falling with the threshold 502. The wound management system determines that the 30% likelihood exceeds the threshold 502. In response, the wound management system 110 determines an action 126 of wearing boots while working to prevent the chef from slipping and falling on wet kitchen floors. The wound management system 110 may recommend to the chef to wear boots while working to reduce the likelihood that the chef will fall while cooking in the kitchen. The wound management system 110 also compares the likelihood of sustaining a self-inflicted wound to the threshold 502. In the example of FIG. 6, the wound management system 110 determines that the 75% likelihood exceeds the threshold 502. In response, the wound management system 110 determines a remedial action 126 of wearing gloves. The wound management system 110 may recommend to the chef to wear protective gloves while working in the kitchen to reduce the likelihood of sustaining a self-inflicted wound (e.g., a burn or a cut). The wound management system 110 compares the likelihood of sustaining a wound from ambulatory conditions to the threshold 502. In the example of FIG. 6, the wound management system 110 compares the 5% likelihood to the threshold 502 and determines that the 5% likelihood does not exceed the threshold 502. In response, the wound management system 110 does not determine a remedial action 126 for the wounds sustained due to ambulatory conditions. In some embodiments, the wound management system 110 may still provide a warning to the chef of the risk of suffering wounds due to ambulatory conditions. Thus, the wound management system can identify an action (e.g. a protective action, a medication, a medical procedure, or another action) to reduce the likelihood that the patient will sustain a wound (e.g., bedsores, sutures, abrasions, lesions, or any other wound). As one example, a patient could be provided with medical treatment (e.g., bandaging, a surgical procedure, a particular medication, or any other suitable treatment), to reduce the likelihood of sustaining a wound. For example, a bedsore, suture, abrasion, or lesion could be identified as less likely to occur if the patient receives identified medication, bandaging, or another medical procedure.

The wound management system 110 may communicate a message to the device 104 of the chef that indicate the determined remedial actions 126. The chef may read the message and perform the remedial actions 126 (e.g., by wearing boots and gloves while working) to reduce the likelihood that the chef will sustain wounds from falling or self-inflicted wounds while working. As a result, the health and well-being of the chef is improved in particular embodiments.

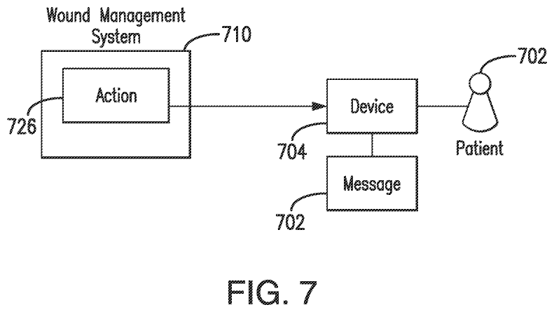

FIG. 7 illustrates an example operation in the system 100 of FIG. 1. Generally, FIG. 7 shows the wound management system 110 communicating determined actions 126 to a device 104 of a patient 102. As discussed previously, the wound management system 110 determines the actions 126 by applying the machine learning model 120 to the health screening data 118 provided by the patient 102 to predict likelihoods that the patient 102 will sustain certain wound types and by comparing those predicted likelihoods to thresholds 502. In some embodiments, the wound management system 110 selects the actions 126 from a database or repository of remedial actions. The wound management system may make these selections based on the wound types and the information in the health screening data 118.

The wound management system 110 communicates the action 126 to the device 104 using a message 702. The message 702 may include the action 126. When the device 104 receives the message 702, the device 104 displays the message 702 to the patient 102. The patient 102 may then read the message 702 to determine the risks of sustaining certain wound types and the remedial actions 126 that the patient 102 may take to reduce the likelihood of sustaining those wound types. The patient 102 may take these actions 126 to reduce the likelihoods, which improves the health and wellbeing of the patient 102.

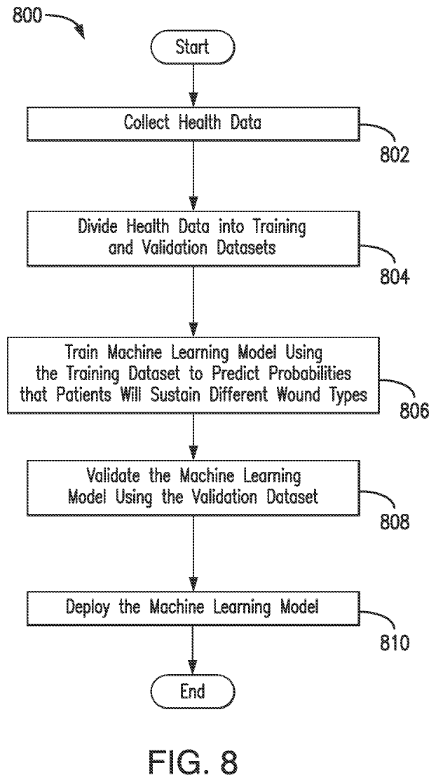

FIG. 8 is a flow chart of an example method 800 performed in the system 100 of FIG. 1. In particular embodiments the wound management system 110 performs the method 800. By performing the method 800, the wound management system 110 trains a machine learning model 120 using health data 112 of previous patients 202.

In block 802, the wound management system 110 collects health data 112. The wound management system 110 may collect or retrieve the health data 112 from a database 108. The health data 112 may have been assembled by collecting information about previous patients 202. For example, a healthcare facility may have collected the health data 112 as it treated the wounds sustained by the previous patients 202. The health data 112 may include information about the previous patients 202 as well as the wound types sustained by the previous patients 202. For example, the health data 112 may include information that identifies the patients 202. The health data 112 may also include information about the wound types sustained by the patients 202, such as the wound type as well as the date on which the wound type was sustained. The health data 112 may also include information about the context in which the wound type was sustained, such as an environment in which or an event at which the wound type was sustained. The health data 112 may further include information about the lifestyle of the patient 202, such as the career of the patient 202 or the habits of the patient 202.

In block 804, the wound management system divides the health data 112 into training data 204 and validation data 206. The wound management system 110 may use any suitable process for dividing the health data 112. For example, the wound management system 110 may divide the health data 112 randomly into the training data 204 and the validation data 206. As another example the wound management system 110 may select a diverse dataset for the training data 204 by clustering the datapoints in the health data 112 into clusters of similar datapoints and then by selecting datapoints from different clusters. As a result, the training data 204 includes datapoints from different clusters, which increases the diversity of the datapoints in the training data 204.

In block 806, the wound management system 110 trains a machine learning model using the training data 204 to predict probabilities that patients will sustain different wound types. For example, the wound management system 110 may have the machine learning model analyze the training data 204 to determine patterns and trends within the training data 204. The machine learning model may also determine sustained wound types that correspond to these detected patterns or trends. In this manner, the machine learning model is trained to determine the likelihood that a particular wound type will be sustained given a particular pattern or trend.

In block 808, the wound management system 110 validates the machine learning model using the validation data 206. The validation data 206 may include datapoints from the health data 112 that were not included in the training data 204. The wound management system 110 may apply the machine learning model to datapoints in the validation data 206 to predict likelihoods that the patients represented by the datapoints in the validation data 206 will sustain particular wound types. The machine learning model predicts these likelihoods, and the wound management system 110 compares these predicted likelihoods with the actual sustained wound types shown in the validation data 206. Based on this comparison, the wound management system 110 determines a loss or accuracy of the machine learning model. If this loss or accuracy is not at an acceptable level, the wound management system 110 may perform another iteration of training for the machine learning model. If the loss or accuracy is at an acceptable level, the wound management system 110 may proceed.

In block 810, the wound management system 110 deploys the machine learning model after the machine learning model has been trained and validated. Stated differently, when the accuracy or loss of the machine learning model is at an acceptable level, the wound management system 110 deploys the machine learning model. In some embodiments the wound management system 110 deploys the machine learning model within the wound management system 110 so that the wound management system 110 may apply the machine learning model to health screening data 118 provided by patients 102. In some embodiments, the wound management system 110 deploys the machine learning model to devices 104 of patients 102. In these embodiments, the devices 104 may apply the machine learning model to health screening data 118 provided by the patients 102. By applying the machine learning model to the health screening data 118, the machine learning model predicts the likelihoods that the patients 102 will sustain different wound types.

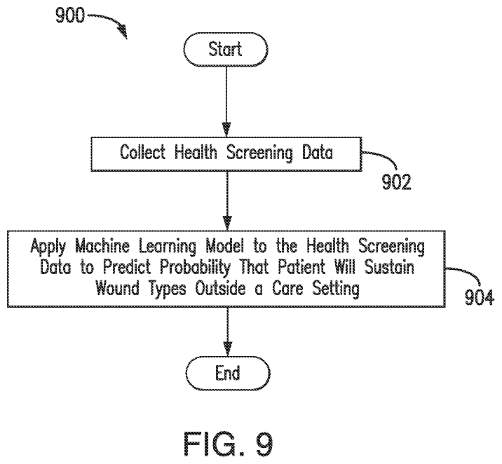

FIG. 9 is a flow chart of an example method 900 performed in the system 100 of FIG. 1. In particular embodiments, the wound management system 110 performs the method 900. By performing the method 900, the wound management system 110 predicts likelihoods or probabilities that a patient 102 will sustain various wound types.

In block 902, the wound management system 110 collects health screening data 118. The health screening data 118 may be provided by the patient 102 using the device 104. For example, the patient 102 may respond to a questionnaire or survey on the device 104. After the patient 102 provides responses, the device 104 communicates the responses to the wound management system as the health screening data 118. The health screening data 118 includes information about the patient 102 that may be helpful in identifying the likelihoods that the patient 102 will sustain different wound types. For example, the health screening data 118 may include information that identifies the patient 102 (e.g., a name and address of the patient 102). The health screening data 118 may include medical information about the patient 102, such as symptoms experienced by the patient 102, allergies of the patient 102, vaccinations or medications taken by the patient 102, previous hospitalizations or operations of the patient 102, and a medical history of other family members of the patient 102. The health screening data 118 may also include information about the lifestyle of the patient 102. For example, the health screening data 118 may include home conditions, work conditions, hobbies, and events of the patient 102. The health screening data 118 may also indicate whether the patient 102 smokes, drinks, wears a seatbelt, or uses recreational drugs. The wound management system 110 may analyze the information in the health screening data 118 to predict likelihoods that the patient 102 will sustain different wound types.

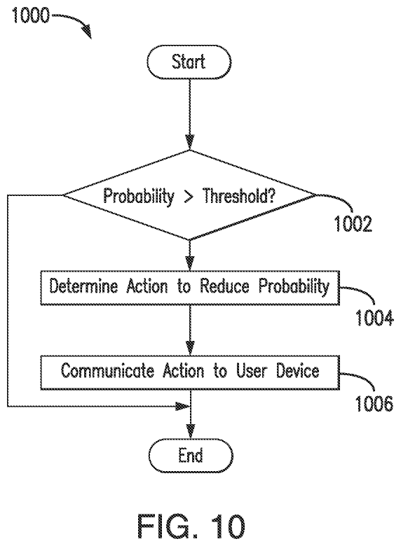

In block 904, the wound management system 110 applies the machine learning model 120 to the health screening data 118 to predict a probability that the patient 102 will sustain a wound type outside a care setting. For example, the wound management system 110 may predict a probability that the patient 102 will sustain a wound type due to a career or habit of the patient 102. For example, the machine learning model 120 may compare the health screening data 118 to the health data 112 of other patients 202 that are similar to the patient 102 to determine the likelihood that the patient 102 will sustain the wound type. After the wound management system 110 applies the machine learning model 120 in block 904, the wound management system 110 may determine an action to recommend by performing the method shown in FIG. 10.

FIG. 10 is a flow chart of an example method 1000 performed in the system 100 of FIG. 1. In particular embodiments, the wound management system 110 performs the method 1000. By performing the method 1000, the wound management system 110 determines actions 126 that a patient 102 may take to reduce the likelihood of sustaining a wound type.

In block 1002, the wound management system 110 determines whether a predicted probability exceeds a threshold 502. The threshold 502 may be set at any suitable level. If the predicted probability that the patient 102 will sustain a particular wound type does not exceed the threshold 502, the wound management system 110 may conclude for that wound type. In some embodiments, the wound management system 110 may generate a warning message about the wound type if the predicted probability for that wound type does not exceed the threshold 502.

If the predicted probability exceeds the threshold 502, the wound management system 110 determines an action 126 to reduce that probability in block 1004. In some embodiments, the wound management system 110 may determine the action 126 from a database or repository of remedial actions 126. These remedial actions 126 may be linked or tied to various wound types in the database or repository. The wound management system 110 may use information from the health screening data 118 along with the predicted wound types to query the database or repository to determine the action 126. After the wound management system 110 determines the action 126, the wound management system 110 communicates the action 126 to a user device 104 in block 1006. For example, the wound management system 110 may communicate a message 702 to the device 104. The message 702 may indicate or include the determined action 126. The device 104 may then display the message 702. When the patient 102 reads the message 702, the patient 102 is informed of the remedial action 126. The patient 102 may then perform or take the action 126 to reduce the likelihood that the patient 102 will sustain the wound type. As a result, the health and well-being of the patient 102 is improved, in certain embodiments.

In certain embodiments, by performing the methods 900 and 1000, the wound management system 110 can predict the likelihood that a patient 102 will sustain a wound outside a care setting. Specifically, conventional processes for determining whether a patient 102 is likely to sustain a wound outside a care setting relied on subjective human judgment and analysis of the patient's 102 information, which resulted in an incomplete analysis of the patient's 102 information and inaccurate predictions. Recommendations and actions taken based on these inaccurate predictions may not actually improve the health and well-being of the patient 102 or reduce the incidences or occurrences of wounds. The wound management system 110, on the other hand, applies a machine learning model to the patient's 102 information to perform a complete analysis of the patient's 102 information, which results in a more accurate prediction of the likelihood that the patient 102 will sustain a wound outside a care setting. The wound management system 110 also provides recommendations based on these more accurate predictions that effects a particular treatment or prophylaxis for preventing or reducing the likelihood of sustaining wounds outside a care setting.

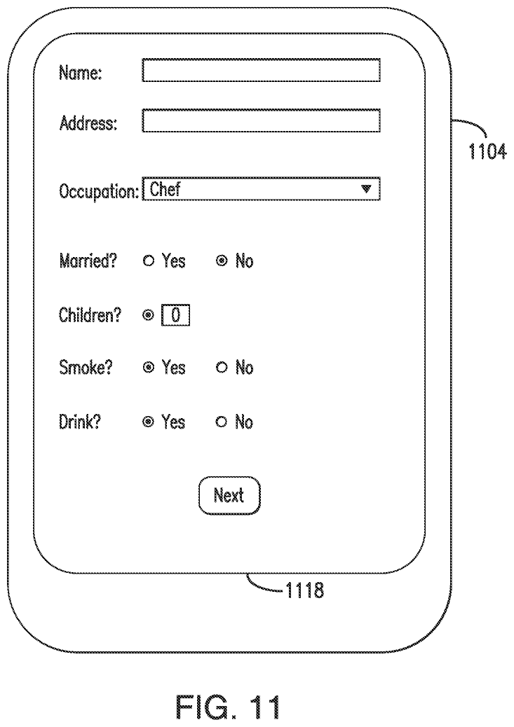

FIG. 11 illustrates an example device 104 in the system 100 of FIG. 1. As seen in FIG. 11, the device 104 presents an interface through which a patient 102 may provide health screening data 118. For example, the interface includes fields in which the patient 102 can provide a name and address. This information may be later used to identify the patient 102. The interface also includes a field that the patient 102 may use to indicate an occupation or career. In the example of FIG. 11, this field is a dropdown list. When the patient 102 selects this field, a list of potential occupations or careers appears. The patient 102 may then select an occupation or career from that list. The wound management system 110 may later use the selected occupation or career to determine likelihoods that the patient 102 will sustain particular wound types. The patient 102 in the example of the FIG. 11 has indicated that the patient 102 is a chef.

The interface also includes fields that the patient 102 may use to indicate the family or home conditions of the patient 102. In the example of FIG. 11, the interface includes fields that the patient 102 may use to indicate whether the patient 102 is married or whether the patient 102 has children. The patient 102 in the example of FIG. 11 has indicated that the patient 102 is not married and has no children. The interface also includes fields that the patient 102 may use to indicate habits of the patient 102. The patient 102 in the example of FIG. 11 has indicated that the patient smokes and drinks.

After the device 104 has collected the information from the patient 102, the device 104 communicates the collected information as health screening data 118 to the wound management system 110. The wound management system 110 may then apply a machine learning model 120 to the health screening data 118 to predict likelihoods that the patient 102 will sustain various wound types (e.g., due to the career or habits of the patient 102). The wound management system 110 may then determine actions 126 that the patient 102 may take to reduce the predicted likelihoods.

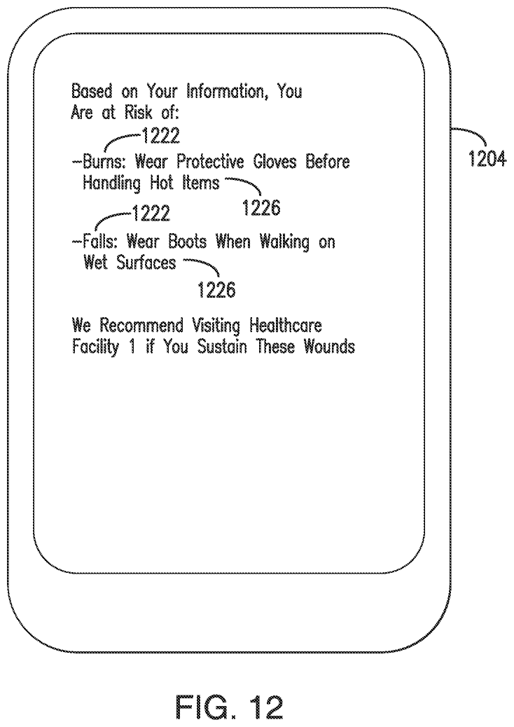

FIG. 12 illustrates an example device 104 in the system 100 of FIG. 1. As seen in FIG. 12, the device 104 displays a message received from the wound management system 110. The message indicates various wound types 122 that the wound management system 110 predicts that the patient 102 is likely to sustain. Additionally, the message includes actions 126 that the wound management system 110 determined will reduce the likelihood that the patient 102 will sustain these wound types 122.

As seen in FIG. 12, the wound management system 110 has determined that the patient 102 is likely to sustain self-inflicted wounds (e.g., burns) and wounds due to falls. The message shown on the device 104 indicates that the patient 102 is likely to experience burns and falls. The wound management system 110 has also determined actions 126 that may be taken by the patient 102 to reduce the likelihood of sustaining burns or falls. As seen in FIG. 12, the message indicates that the patient 102 should wear protective gloves before handling hot items to reduce the likelihood of experiencing burns. Additionally, the message indicates that the patient 102 should wear boots when walking on wet surfaces to reduce the likelihood of falls. The device 104 displays these actions 126 to the patient 102. The patient 102 may take or perform these actions to reduce the likelihood of sustaining burns and falls, which improves the health and wellbeing of the patient 102 in particular embodiments.

In some embodiments, the message also includes a recommendation of a healthcare facility for treating the predicted wound types. The wound management system 110 may determine the healthcare facility by first determining healthcare facilities that are near the patient 102 based on the address provided by the patient 102 in the health screening data 118. The wound management system 110 may then determine, based on statistics about the healthcare facilities, the healthcare facility that is best suited for treating the predicted wound types. The wound management system 110 then includes that healthcare facility in the message communicated to the device 104. By providing this information to the patient 102, the patient 102 may know which healthcare facility to visit if the patient 102 sustains any of the predicted wound types.

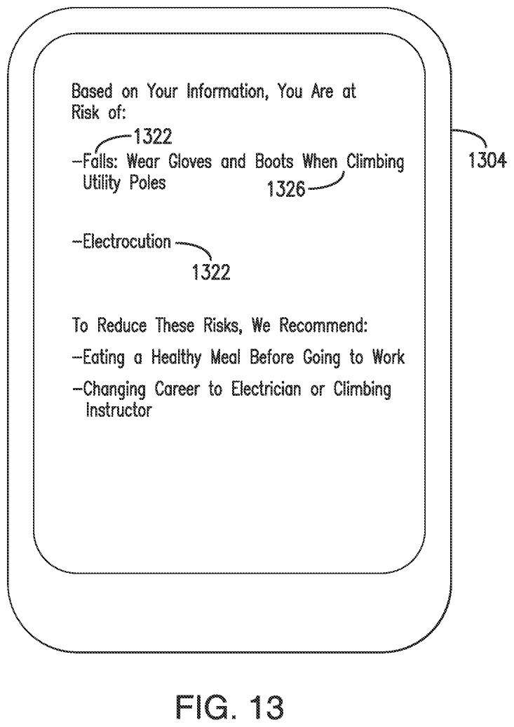

FIG. 13 illustrates an example device 104 in the system 100 of FIG. 1. Generally, the device 104 in the example of FIG. 13 is a device 104 of a utility maintenance provider who enjoys rock climbing. The device 104 may receive a message from the wound management system 110 indicating wound types that the patient 102 is likely to sustain. In the example of FIG. 13, the message indicates that the patient 102 is likely to sustain wounds from falls or wounds from electrocution.

The message also includes a remedial action 126 that the patient 102 can take to reduce the likelihood that the patient 102 will sustain a wound from a fall. As seen in FIG. 13, the action 126 includes wearing gloves and boots when climbing utility poles. The device 104 presents the message that includes this action 126 to let the patient 102 know that the patient 102 should wear gloves and boots when climbing utility poles to reduce the likelihood of sustaining a wound from falls. The patient 102 may perform or take the action 126 to reduce the likelihood of falling, which improves the health and well-being of the patient 102 in certain embodiments. Additionally, as seen in FIG. 13, no action 126 accompanies the electrocution wound type. As discussed previously, the wound management system 110 may not have determined an action 126 corresponding to the electrocution wound type because the predicted likelihood for the electrocution wound type did not exceed a threshold 502. In response, the wound management system 110 provides a warning of the electrocution wound type but does not provide a remedial action 126 to reduce the likelihood of the electrocution wound type.

The wound management system 110 also provides recommendations for changes to the patients 102 lifestyle that will reduce the risks of sustaining the predicted wound types. In the example of FIG. 13, the wound management system 110 determined that eating a healthy meal before going to work may provide the patient 102 more energy while climbing utility poles, which may reduce the likelihood of falling. Moreover, eating a healthy meal may keep the patient 102 while working, which reduces the likelihood of electrocution. Additionally, the wound management system 110 determined that changing careers to electrician or climbing instructor may reduce the likelihood that the patient 102 will sustain the predicted wound types. As discussed previously, the wound management system 110 may have determined these actions 126 or lifestyle changes by analyzing other clusters of patients 202 that have a low likelihood of sustaining the predicted wound types of the patient 102.

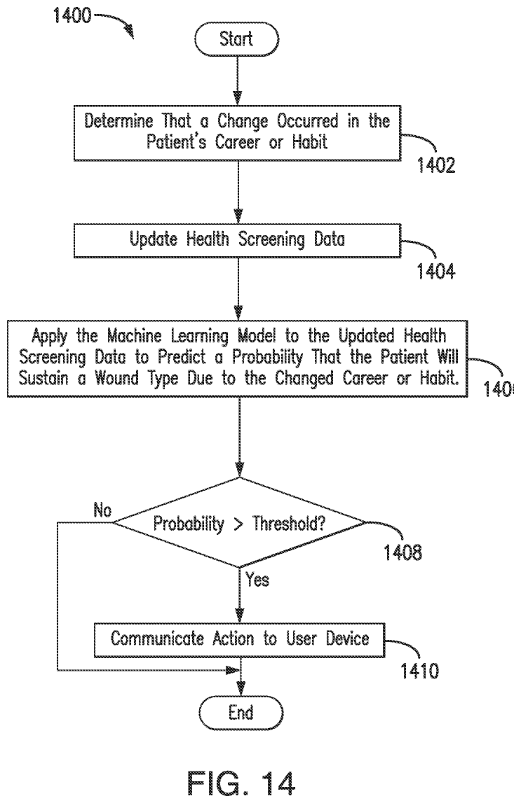

FIG. 14 is a flow chart of an example method 1400 performed in the system 100 of FIG. 1. In particular embodiments, the wound management system 110 performs the method 1400. By performing the method 1400 the wound management system 110 updates or changes the predicated likelihoods for a patient 102.

In block 1402, the wound management system 110 determines that a change occurred in the patient's career or habit. The wound management system 110 may determine the change by analyzing the activities of the patient 102 when carrying the device 104. The wound management system 110 may determine that the patient 102 is traveling to different locations or engaging in different activities. For example, the wound management system 110 may track the location of the device 104 to determine that the patient 102 is traveling to different locations not pertaining to the patients 102 old careers or habits. As another example, the wound management system 110 may examine a social media feed of the patient 102 to determine that the patient 102 is engaging in different activities or hobbies. As yet another example, the patient 102 may input the changes into the device 104 so that the wound management system 110 determines that the changes are occurring.

In block 1404, the wound management system 110 updates the health screening data 118 based on the changes determined in block 1402. For example, the wound management system 110 may update the career, conditions, or habits of the patient 102 in the health screening data 118.

In block 1406, the wound management system 110 applies the machine learning model 120 to the updated health screening data 118 to predict a probability that the patient 102 will sustain a wound type 122 due to the changed career or habit. Stated differently, the wound management system 110 applies the machine learning model 120 to the updated data to determine a likelihood that the patient 102 will sustain a particular wound type 122. The machine learning model 120 may then determine from the health data 112 of these other patients 202 the likelihood that the patient 102 is likely to sustain the wound type 122.

In block 1408, the wound management system 110 determines whether the predicted probability exceeds the threshold 502. If the predicted probability does not exceed the threshold 502, the wound management system 110 may conclude. In some embodiments, the wound management system 110 still provides a warning message of the predicted wound type 122 when the predicted probability does not exceed the threshold 502. If the predicted probability exceeds the threshold 502, the wound management system 110 determines and communicates an action 126 to the device 104 of the patient 102 in block 1410. The action 126 may be a remedial action that if taken by the patient 102 reduces the likelihood that the patient 102 will sustain the predicated wound type 122. In this manner, the wound management system 110 takes a proactive approach to reduce the likelihood that the patient 102 will sustain a predicted wound type, which improves the health and safety of the patient 102 in particular embodiments.

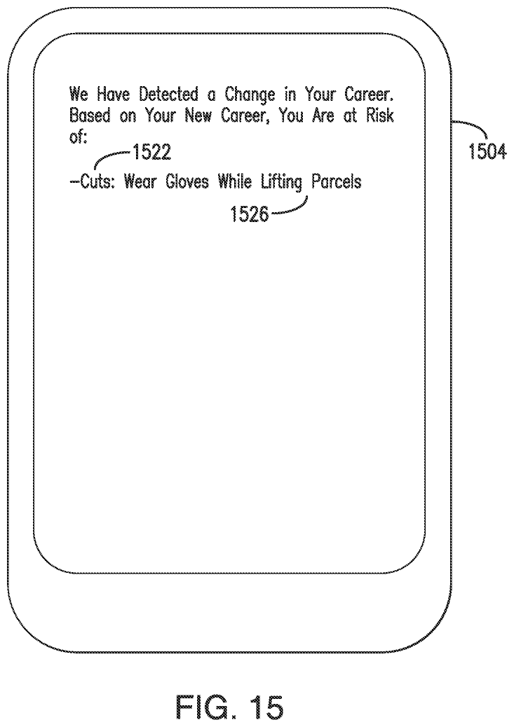

FIG. 15 illustrates an example device 104 in the system 100 of FIG. 1. Generally, the device 104 in FIG. 15 shows a message that the wound management system 110 provides in response to detecting an update to the health screening data 118 of a patient 102. As seen in FIG. 15, the message indicates that a change has been detected in the career of the patient 102. Based on the change in careers, the patient 102 is at risk of experiencing cuts. Because the wound management system 110 determines that the predicated likelihood of sustaining a cut exceeds the threshold 502, the wound management system 110 provides a remedial action 126 that reduces the likelihood that the patient 102 will sustain a cut. In the example of FIG. 15, the message indicates that the action 126 is to wear gloves while lifting parcels. If the patient 102 wears gloves while lifting parcels, the patient 102 reduces the likelihood that the patient 102 will sustain a cut.

In summary, a wound management system 110 uses machine learning to predict whether a patient 102 is likely to sustain different wound types 122 based on information about the patient's 102 life. For example, the wound management system 110 may predict that a patient 102 is more likely to sustain cuts or burns if the patient 102 is a chef. As another example, the wound management system 110 may predict that the patient 102 is more likely to sustain wounds from falling if the patient 102 enjoys rock climbing. The wound management system 110 may also prevent the predicted wound types 122 from occurring by recommending actions 126 that the patient 102 can take to reduce the likelihood of sustaining the wound types 122. In this manner, the wound management system 110 provides a proactive approach towards wound treatment, which improves the health and well-being of the patient 102, in certain embodiments.

### Example Clauses

Implementation examples are described in the following numbered clauses:

Clause 1: A method includes collecting data relating to a patient's health and applying a machine learning model to the data relating to the patient's health to predict a first probability that the patient will sustain a first wound type outside of a care setting. The method also includes, in response to determining that the first probability exceeds a threshold, determining an action that reduces the first probability and communicating, to the patient, a message indicating the action should be taken to reduce the first probability that the patient will sustain the first wound type.

Clause 2: The method of Clause 1, further including collecting a dataset indicating past physical wounds sustained by different patients, dividing the dataset into a training dataset and a validation dataset, training the machine learning model using the training dataset, and validating the machine learning model using the validation dataset after the machine learning model is trained.

Clause 3: The method of any of Clauses 1-2, wherein the dataset indicates a plurality of wound types for the past physical wounds.

Clause 4: The method of any of Clauses 1-3, wherein the plurality of wound types for the past physical wounds comprise wounds sustained during a fall, self-inflicted wounds, and wounds from ambulatory conditions.

Clause 5: The method of any of Clauses 1-4, wherein the data relating to the patient's health comprises a career or a habit of the patient and the first probability indicates a likelihood that the patient will sustain the first wound type due to the career or habit.

Clause 6: The method of any of Clauses 1-5, wherein the action comprises wearing a type of apparel while engaging in the career or the habit.

Clause 7: The method of any of Clauses 1-6, wherein the action comprises changing the career of the patient.

Clause 8: The method of any of Clauses 1-7, further including predicting a second probability that the patient will sustain a second wound type due to the career or habit and in response to determining that the second probability does not exceed the threshold, communicating, to the patient, a message warning of the second wound type.

Clause 9: The method of any of Clauses 1-8, further including, in response to determining that a change in the career or the habit has occurred, updating the data relating to the patient's health to produce updated data and applying the machine learning model to the updated data to predict a second probability that the patient will sustain a second wound type due to the change.

Clause 10: The method of any of Clauses 1-9, wherein the message further indicates a healthcare facility to treat the first wound type.

Clause 11: The method of any of Clauses 1-10, wherein the first wound type encompasses a plurality of wounds.

Clause 12: An apparatus including a memory and a hardware processor communicatively coupled to the memory configured to perform a method in accordance with any one of Clauses 1-11.

Clause 13: A non-transitory computer-readable medium including instructions that, when executed by a processor, cause the processor to perform a method in accordance with any one of Clauses 1-11.

### ADDITIONAL CONSIDERATIONS

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

In the preceding, reference is made to embodiments presented in this disclosure. However, the scope of the present disclosure is not limited to specific described embodiments. Instead, any combination of the described features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the preceding aspects, features, embodiments and advantages are merely illustrative and are not considered elements or limitations of the appended claims except where explicitly recited in a claim(s).

As will be appreciated by one skilled in the art, the embodiments disclosed herein may be embodied as a system, method or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium is any tangible medium that can contain, or store a program for use by or in connection with an instruction execution system, apparatus or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems) and computer program products according to embodiments presented in this disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality and operation of possible implementations of systems, methods and computer program products according to various embodiments. In this regard, each block in the flowchart or block diagrams may represent a module, segment or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

What is claimed is:

1. A method comprising:
collecting data relating to a patient's health;
applying a machine learning model to the data relating to the patient's health to predict a first probability that the patient will sustain a first wound type outside of a care setting;
in response to determining that the first probability exceeds a threshold, determining an action that reduces the first probability; and
communicating, to the patient, a message indicating the action should be taken to reduce the first probability that the patient will sustain the first wound type.

2. The method of claim 1, further comprising:
collecting a dataset indicating past physical wounds sustained by different patients;
dividing the dataset into a training dataset and a validation dataset;
training the machine learning model using the training dataset; and
validating the machine learning model using the validation dataset after the machine learning model is trained.

3. The method of claim 2, wherein:
the dataset indicates a plurality of wound types for the past physical wounds.

4. The method of claim 3, wherein:
the plurality of wound types for the past physical wounds comprises wounds sustained during a fall, self-inflicted wounds, and wounds from ambulatory conditions.

5. The method of claim 1, wherein:
the data relating to the patient's health comprises a career or a habit of the patient; and
the first probability indicates a likelihood that the patient will sustain the first wound type due to the career or habit.

6. The method of claim 5, wherein:
the action comprises wearing a type of apparel while engaging in the career or the habit.

7. The method of claim 5, wherein:
the action comprises changing the career of the patient.

8. The method of claim 5, further comprising:
predicting a second probability that the patient will sustain a second wound type due to the career or habit; and
in response to determining that the second probability does not exceed the threshold, communicating, to the patient, a message warning of the second wound type.

9. The method of claim 5, further comprising:
in response to determining that a change in the career or the habit has occurred, updating the data relating to the patient's health to produce updated data; and
applying the machine learning model to the updated data to predict a second probability that the patient will sustain a second wound type due to the change.

10. The method of claim 1, wherein:
the message further indicates a healthcare facility to treat the first wound type.

11. The method of claim 1, wherein the first wound type encompasses a plurality of wounds.

12. An apparatus comprising:
a memory; and
a hardware processor communicatively coupled to the memory, the hardware processor configured to:
collect data relating to a patient's health;
apply a machine learning model to the data relating to the patient's health to predict a first probability that the patient will sustain a first wound type outside a care setting;
in response to determining that the first probability exceeds a threshold, determine an action that reduces the first probability; and
communicate, to the patient, a message indicating the action should be taken to reduce the first probability that the patient will sustain the first wound type.

13. The apparatus of claim 12, wherein the hardware processor is further configured to:
collect a dataset indicating past physical wounds sustained by different patients;
divide the dataset into a training dataset and a validation dataset;
train the machine learning model using the training dataset; and
validate the machine learning model using the validation dataset after the machine learning model is trained.

14. The apparatus of claim 13, wherein:
the dataset indicates a plurality of wound types for the past physical wounds.

15. The apparatus of claim 14, wherein:
the plurality of wound types for the past physical wounds comprises wounds sustained during a fall, self-inflicted wounds, and wounds from ambulatory conditions.

16. The apparatus of claim 12, wherein:
the data relating to the patient's health comprises a career or a habit of the patient; and
the first probability indicates a likelihood that the patient will sustain the first wound type due to the career or habit.

17. The apparatus of claim 16, wherein:
the action comprises wearing a type of apparel while engaging in the career or the habit.

18. The apparatus of claim 16, wherein:
the action comprises changing the career of the patient.

19. The apparatus of claim 16, further comprising:
predicting a second probability that the patient will sustain a second wound type due to the career or habit; and
in response to determining that the second probability does not exceed the threshold, communicating, to the patient, a message warning of the second wound type.

20. A non-transitory computer-readable medium comprising instructions that, when executed by a processor, cause the processor to:
collect data relating to a patient's health;
apply a machine learning model to the data relating to the patient's health to predict a first probability that the patient will sustain a first wound type outside of a care setting;
in response to determining that the first probability exceeds a threshold, determine an action that reduces the first probability; and
communicate, to the patient, a message indicating the action should be taken to reduce the first probability that the patient will sustain the first wound type.
